(19) United States
(12) Patent Application Publication
(10) Pub. No.: US 2015/0190407 A1
(43) Pub. Date: Jul. 9, 2015

(54) METHODS FOR TREATMENT OF POSTOPERATIVE INFLAMMATION WITH REDUCED INTRAOCULAR PRESSURE

(71) Applicant: Insite Vision Incorporated, Alameda, CA (US)

(72) Inventors: Kamran HOSSEINI, Hayward, CA (US); Lyle M. BOWMAN, Pleasanton, CA (US)

(73) Assignee: INSITE VISION INCORPORATED, Alameda, CA (US)

(21) Appl. No.: 14/149,743

(22) Filed: Jan. 7, 2014

(51) Int. Cl. A61K 31/573; A61K 31/568; A61K 47/36; A61K 9/00; A61K 47/32

(52) U.S. Cl. CPC A61K 31/573; A61K 9/0048; A61K 47/32; A61K 47/36; A61K 31/568

(57) ABSTRACT

Methods of treating a subject after ocular surgery are disclosed. The methods include administering to an eye of a subject in need thereof a composition comprising at least one corticosteroid or an ophthalmically acceptable salt thereof in an ophthalmically acceptable vehicle that can provide a sustained release of the at least one corticosteroid or an ophthalmically acceptable salt thereof. Advantageously, the composition does not result in a statistically significant elevation in intraocular pressure in a statistically significant number of subject eyes when administered twice daily over a period of two weeks.

### TECHNICAL FIELD

The present disclosure relates to a method of treating postoperative inflammatory reaction following ocular surgery such as cataract surgery by administering to a subject in need thereof a composition including one or more corticosteroid in a sustained release vehicle.

### BACKGROUND

Topical corticosteroids (including glucocorticoids) are commonly used as a routine treatment following postoperative surgery to reduce inflammatory reaction thereto and other possible complications arising from ocular surgery. However, it is widely known that certain corticosteroids such as medrysone, fluorometholone, dexamethasone, prednisolone, and their esters adversely cause elevation of intraocular pressure. See Mindel et al. “Comparative Ocular Pressure Elevation by Medrysone, Fluorometholone, and Dexamethasone Phosphate”, Arch. Ophthalmol. 1980:98:1577-78; Laurell et al., “Effects of dexamethasone, diclofenac, or placebo on the inflammatory response after cataract surgery”, Br. J. Ophthamol 2002:86:1380-1384.

Further, the risk of intraocular pressure elevation increases with the duration of use of the corticosteroid. For example, Mindel et al. at 1578 show an increase of intraocular pressure with use of dexamethasone phosphate of about 1-2 mmHg after one week, 3-4 mmHg after two weeks and 5-6 mmHg after six weeks. Laurell et al. at 1382 report that after 8 days of treating group I subjects with dexamethasone phosphate for inflammation following ocular surgery, the median intraocular pressure was significantly higher than subjects (group III) receiving a placebo (saline 0.9%).

Despite the medicinal benefits of treating postoperative inflammatory reaction following ocular surgery with a corticosteroid, the use of such a corticosteroid can adversely affect the intraocular pressure, particularly when the corticosteroid is administered over a period of one or more weeks. Hence a need exists to more safely and effectively treat postoperative inflammatory reaction following ocular surgery with a corticosteroid.

### SUMMARY OF THE DISCLOSURE

An advantage of the present disclosure is use of steroids for treatment in the eye following ocular surgery for long periods of time without causing the side effect of high intraocular pressure, which can damage the eye.

These and other advantages are satisfied, at least in part, by a method of treating a subject after ocular surgery, e.g., cataract surgery. The method comprising administering to an eye of a subject in need of such treatment a composition comprising at least one corticosteroid or an ophthalmically acceptable salt thereof in an ophthalmically acceptable vehicle that can provide a sustained release of the at least one corticosteroid or an ophthalmically acceptable salt thereof.

Another aspect of the present disclosure include methods of treating a patient after ocular surgery, the method comprising administering to an eye of a subject in need thereof a composition comprising dexamethasone or a pharmaceutically acceptable salt thereof in an ophthalmically acceptable vehicle that can provide a sustained release of the dexamethasone or an ophthalmically acceptable salt thereof.

Advantageously, the compositions of the present disclosure do not result in a statistically significant elevation in intraocular pressure in a statistically significant number of subject eyes when administered twice daily over a period of two weeks. The composition can be administered for example one to four times a day for a period of one to six weeks.

Embodiments of the present disclosure include wherein the vehicle comprises an aqueous suspension including a carboxy-containing polymer, e.g., a polycarbophil, in an amount of about 0.5% to about 1.5% by weight of the composition and optionally a cationic polymer, e.g., chitosan, and wherein a viscosity of the composition is in the range of about 1,000 to about 2,000 cps and wherein the at least one corticosteroid, e.g., dexamethasone, or an ophthalmically acceptable salt thereof is in an amount of about 0.01% to about 1% by weight of the composition.

Additional advantages of the present invention will become readily apparent to those skilled in this art from the following detailed description, wherein only the preferred embodiment of the invention is shown and described, simply by way of illustration of the best mode contemplated of carrying out the invention. As will be realized, the invention is capable of other and different embodiments, and its several details are capable of modifications in various obvious respects, all without departing from the invention. Accordingly, the description is to be regarded as illustrative in nature, and not as restrictive.

### DETAILED DESCRIPTION OF THE DISCLOSURE

The present disclosure relates to the surprising discovery that a corticosteroid, e.g. dexamethasone, or an ophthalmically acceptable salt thereof can be administered in a composition to treat an eye condition of a subject characterized by inflammation and/or pain in an effective amount with minimal to no elevation in intraocular pressure when the corticosteroid, or an ophthalmically acceptable salt thereof is administered in an ophthalmically acceptable vehicle that can provide a sustained release of the medicament. It is widely known that corticosteroids adversely cause elevation of intraocular pressure (TOP) and the IOP increases with the duration and an increase in the frequency of use of the corticosteroid. However, such medicaments are useful for reducing ocular inflammation following ocular surgery such as after cataract surgery. The methods of the present disclosure allow treating a postoperative ocular inflammatory reaction with a corticosteroid, or an ophthalmically acceptable salt thereof without the concomitant adverse effects of IOP even for treatment periods of several weeks.

Methods of practicing the present disclosure include, for example, treating a subject after ocular surgery by administering to an eye of the subject in need thereof a composition comprising at least one corticosteroid or an ophthalmically acceptable salt thereof in an ophthalmically acceptable vehicle that can provide a sustained release of the at least one corticosteroid or an ophthalmically acceptable salt thereof. Advantageously, the composition does not result in a statistically significant elevation in intraocular pressure in a statistically significant number of subject eyes when administered twice daily over a period of two weeks.

While the compositions employed in the present disclosure do not result in a statistically significant elevation in intraocular pressure in a statistically significant number of subject eyes when administered twice daily over a period of two weeks, the compositions of the present disclosure need not be so administered. As is known in the art, the administration of a medicament is not necessarily limited to its maximum tolerability or tested conditions. Indeed, medicaments are frequently administered below their tolerable limit, and, when appropriate well above their tested conditions. In accordance with the present disclosure, the method of treating the subject includes administering the composition, the method of treating the subject includes administering the composition at least one, two, three to four times a day or more for a period of at least one, two, four, six weeks or longer.

The compositions useful in practicing the methods of present disclosure do not cause a statistically significant elevation in intraocular pressure in a statistically significant number of subject eyes when administered twice daily over a period of two weeks. Determining whether a composition results in statistically significant elevation in intraocular pressure is readily known in the ophthalmic arts. See, e.g., Mindel et al. “Comparative Ocular Pressure Elevation by Medrysone, Fluorometholone, and Dexamethasone Phosphate”, Arch. Ophthalmol. 1980:98:1577-78. Such determinations can be made, for example, by using a standard tonometer with a statistically significant number of appropriate subject eyes and making the measurement before the administration period and at the end of the administration period. In one aspect of the present disclosure, a statistically significant elevation in intraocular pressure can be determined by administering a composition to at least 12 eyes twice daily over a two week period. Other embodiments of the present disclosure include administering compositions at least daily, twice daily, three times daily, four times daily over a period of one, two, three, four, six or more weeks.

In practicing methods of the present disclosure, the subject can be treated following surgical trauma, e.g., ocular surgery, for conditions related to inflammation and/or pain, etc. Embodiments of the present disclosure include treating a subject following ocular surgery such as cataract surgery, LASIK, photorefractive surgery, excimer laser phototherapeutic keratectomy conditions associated with refractive surgery, etc. In one aspect of the present disclosure, a subject is treated following cataract surgery for inflammation and/or pain by administering to the eye or eyes of the subject in need thereof a composition comprising at least one corticosteroid, e.g., dexamethasone, or an ophthalmically acceptable salt thereof in an ophthalmically acceptable vehicle that can provide a sustained release of the at least one corticosteroid or an ophthalmically acceptable salt thereof. The administration can be for at least one to four times a day for a period of at least one to six weeks or longer. Advantageously, the composition does not cause a statistically significant elevation in intraocular pressure in a statistically significant number of subject eyes for the administration dose or period.

Corticosteroids that typically cause an elevation in IOP can be used in the composition of the present disclosure such that administering the corticosteroid results in minimal to no statistically significant IOP. Such corticosteroids include, for example, corticosterone, medrysone, fluorometholone their esters and ophthalmically acceptable salt thereof. The corticosteroids of the present disclosure also includes glucocorticoids their esters and ophthalmically acceptable salt thereof. Such glucocorticoids include, for example, hydrocortisone, cortisone acetate, prednisone, prednisolone, methylprednisolone, dexamethasone, betamethasone, triamcinolone, and beclomethasone, fluorometholone. Other glucocorticoids useful in the method for treating blepharitis include, for example, 21-acetoxypregnenolone, alclometasone, algestone, amcinonide, budesonide, chloroprednisone, clobetasol, clobetasone, clocortolone, cloprednol, corticosterone, cortisone, cortivazol, deflazacort, desonide, desoximetasone, diflorasone, diflucortolone, difluprednate, enoxolone, fluazacort, flucloronide, flumethasone, flunisolide, fluocinolone acetonide, fluocinonide, fluocortin butyl, fluocortolone, fluperolone acetate, fluprednidene acetate, fluprednisolone, flurandrenolide, fluticasone propionate, formocortal, halcinonide, halobetasol propionate, halometasone, halopredone acetate, hydrocortarnate, loteprednol etabonate, mazipredone, medrysone, meprednisone, mometasone furoate, paramethasone, prednicarbate, prednisolone 25-diethylamino-acetate, prednisolone sodium phosphate, prednival, prednylidene, rimexolone, tixocortol, triamcinolone acetonide, triamcinolone benetonide, triamcinol one hexacetonide, their ophthalmically acceptable salts, combinations thereof, and mixtures thereof. In one embodiment, the glucocorticoid includes dexamethasone, prednisone, prednisolone, methylprednisolone, medrysone, triamcinolone, loteprednol etabonate, ophthalmically acceptable salts thereof, combinations thereof, and mixtures thereof.

The corticosteroids and glucocorticoids useful for the present disclosure include the ester derivatives. For example dexamethasone esters include dexamethasone phosphate, dexamethasone acetate, dexamethasone dimethylbutyrate, dexamethasone trimethylacetate, dexamethasone dipropionate, dexamethasone acefurate, etc. When used herein, the term's corticosteroid, glucocorticoid or any particular corticosteroid, or glucocorticoid includes their esters unless otherwise stated. Hence, the term dexamethasone includes dexamethasone (alcohol) and its esters.

Ophthalmically acceptable vehicles that can provide a sustained release of the at least one corticosteroid or an ophthalmically acceptable salt thereof include, for example, an aqueous suspension including at least one lightly cross-linked carboxy-containing polymer, e.g., a polycarbophil, Carbopol, or Noveon polymer. In certain embodiments, the composition further includes at least a cationic polymer, e.g., chitosan.

The ophthalmic vehicle preferably has desirable rheological properties that are conducive to medicament delivery into the eye and provide corneal retention. The vehicle uses a combination of an anionic carboxy-containing polymer in conjunction with a substantially smaller amount of a second polymer, for example, a cationic polymer. The second polymer is included at a sufficiently low concentration such that the particles of the carboxy-containing polymer remain suspended, yet when combined with the second polymer, the resulting vehicle has higher viscosity than the vehicle with the carboxy-containing polymer alone. The vehicle disclosed herein has the property that, when combined with tear fluid, its viscosity increases due to the higher pH of tear fluid. The resultant viscosity provides a means by which to increase the efficiency of medicament delivery and corneal retention.

The ophthalmically acceptable vehicle useful for the present disclosure preferably has suitable mucoadhesive properties that can facilitate the absorption of the medicament by increasing the contact time of the drug with the ocular mucosa. Interactions between the vehicle and the ocular mucosa can include Van der Waals attractive forces, hydrogen bonding, and electrostatic interactions between the mucins of the ocular mucosa and the carboxy-containing polymer and the second polymer. Together, these forces can increase the residence time of a medicament in the eye. An additional benefit of the ophthalmically acceptable vehicle disclosed herein, is the ability to provide the medicament in a sustained release manner.

Ophthalmically acceptable vehicles useful for the present disclosure includes an aqueous suspension containing from about 0.1% to about 6.5%, e.g., from about 0.5% to about 1.5%, by weight, based on the total weight of the suspension, of a carboxyl-containing polymer prepared by polymerizing one or more carboxyl-containing monoethylenically unsaturated monomers and less than about 5% by weight of a crosslinking agent. The weight percentages of monomers are based on the total weight of monomers polymerized. The carboxyl-containing polymer has an average particle size of not more than about 50 μm in equivalent spherical diameter and is lightly cross-linked.

The vehicle further includes a second polymer, such as a cationic polymer, added in sufficient amount to increase the vehicle viscosity without the loss of polymer particle suspension, while still allowing the vehicle to be administered to the eye in drop form. Upon contact of the lower pH vehicle with higher pH tear fluid, the vehicle rapidly gels to a greater viscosity and therefore can remain in the eye for sustained release of a medicament contained within the vehicle.

As used herein an “ophthalmically acceptable vehicle” is one which allows delivery of a medicament to the eye and/or eyelids, to treat an ocular condition without deleterious effects on the eye. An ophthalmically acceptable vehicle is one that can maintain proper intraocular pressure and provide solutions of medicaments that are isotonic, mildly hypotonic, or mildly hypertonic. To maintain such conditions one can include various non-ionic osmolality-adjusting compounds such as polyhydric alcohols, including for example, glycerol, mannitol, sorbitol, or propylene glycol. Alternatively, osmolality adjusting compounds can include ionic salts such as sodium or potassium chloride. An ophthalmically acceptable vehicle can also include buffers to adjust the vehicle to an acceptable pH, which can range from about 3 to 6.5, and in some embodiments from about 4 to 8, including any pH in between. Such buffer systems include, but not limited to, acetate buffers, citrate buffers, phosphate buffers, borate buffers and mixtures thereof. Specific buffer components useful in the present compositions include, but not limited to, citric acid/sodium citrate, boric acid, sodium borate, sodium phosphates, including mono, di- and tri-basic phosphates, such as sodium phosphate monobasic monohydrate and sodium phosphate dibasic heptahydrate, and mixtures thereof. It should be noted that any other suitable ophthalmically acceptable buffer components can be employed to maintain the pH of the ophthalmic formulation so that the ophthalmic formulation is provided with an acceptable pH, and the foregoing buffer components are merely exemplary examples of such buffer components.

As used herein, the term “carboxyl-containing polymer” refers to a polymer that contains the carboxylic acid functional group. This functional group can be substantially ionized, for example, and exist as a carboxylate anion (COO), rendering the polymer negatively charged. In the context of the ophthalmically acceptable vehicle, the degree of ionization can depend on the pH, which is mediated by any buffer system, and the presence other components in the vehicle that contain Lewis basic atoms, such as an amine-functionalized polymer. A Lewis base is donor of a pair of electrons and as such, is capable of accepting hydrogen ion (H⁺) from a carboxyl group (COOH).

As used herein, the term “cationic polymer” refers to a positively-charged, amine-functionalized polymer. The polymer contains nitrogen atoms that are quaternized or capable of being quaternized upon adjustment to a sufficiently low pH and/or in the presence of a proton donor, such as the carboxyl containing polymer, or other Lewis acid (i.e. an electron pair acceptor). A quaternized nitrogen atom is a nitrogen atom engaged in bonding to four other atoms, thus causing nitrogen to have a net formal charge of plus one (+1). Examples of nitrogen atoms carrying positive charge include, but not limited to, NR₄⁺, NR₃H⁺, NR₂H⁺, NRH₂⁺, wherein R can represent any atom or group of atoms bonded to nitrogen.

As used herein “viscosity” refers to a fluid's resistance to flow. The unit of viscosity is dyne second per square centimeter [dyne·s/cm²], or poise [P]. This type of viscosity is also called dynamic viscosity, absolute viscosity, or simple viscosity. This is distinguished from kinematic viscosity which is the ratio of the viscosity of a fluid to its density.

As used herein “mucoadhesive” or “mucoadhesion” refers to the ability of the ophthalmically acceptable vehicle to adhere to the ocular mucosa. Mucoadhesive agents used in the composition include carboxy-containing polymers capable of forming hydrogen bonds. Mucoadhesion can depend on pH and the density of hydrogen bonding groups. In the vehicle of the present composition, the density of cross-linking in the carboxy-containing polymer can affect mucoadhesion. Thus, a lightly cross-linked polymer system has sufficient flexibility to form multiple hydrogen bonds, making it a good mucoadhesive agent. Another vehicle component that can affect mucoadhesion is the second polymer, which can interact with the carboxy-containing polymer, as explained further below.

As used herein, “administered to the eye” means that an ophthalmically acceptable vehicle, along with a medicament, is in the form of an eye drop that can be applied directly to the surface of the eye and/or in the eyelid margins, such administration techniques being familiar to persons skilled in the art.

As used herein, “an effective amount” when used in connection with treating an ocular condition is intended to qualify the amount of a medicament used in the treatment of a particular ocular condition. This amount will achieve the goal of preventing, reducing, or eliminating the ocular condition. An effective amount depends on the particular medicament to be administered, although ophthalmic formulations can include, for example, from about 0.01% to about 5.0% by weight, while in other embodiments the active ingredient is present in a range from about 0.05% to about 1%, e.g., about 0.08% to about 0.15% by weight. 0.01 mg/ml to 100 mg/ml per dose in one embodiment and from about 1 to 50 mg/ml dose in another embodiment. An “effective amount” can include a dose regimen once per day, twice per day, thrice per day, and so on.

As used herein “medicament” refers to the primary compound responsible for reducing, preventing, or eliminating the clinical signs and symptoms of an ocular condition.

As used herein “an ophthalmically acceptable salt” will include those that exhibit no deleterious effects on the eye as well as being compatible with the active ingredient itself and the components of the ophthalmically acceptable vehicle. Salts or zwitterionic forms of a medicament can be water or oil-soluble or dispersible. The salts can be prepared during the final isolation and purification of the medicament or separately by adjusting the pH of the appropriate medicament formulation with a suitable acid or base.

In some embodiments, the ophthalmically acceptable vehicle includes carboxy-containing polymers in conjunction with a cationic polymer added in sufficient amount to increase the vehicle viscosity, while still allowing the carboxy-containing polymer particles to remain suspended. The vehicle can be in the form of a gel or liquid drops which release a medicament over time when administered to the eye. The carboxy-containing polymer is about 0.1 to about 6.5% in some embodiments, and, in other embodiments about 0.5% to about 1.5%, e.g., about 1.0 to about 1.3%, by weight based on the total weight of the suspension of a cross-linked carboxy-containing polymer. Suitable carboxy-containing polymers are described, for example, in U.S. Pat. Nos. 5,192,535 and 8,501,800 which are hereby incorporated in their entirety by reference and include lightly crosslinked carboxy-containing polymers such as polycarbophil (Noveon AA-1, or CARBOPOLS® polymers available from Lubrizol Corp. Wickliffe, Ohio). A carboxy-containing polymer system known by the tradename DURASITE® is a polycarbophil-based sustained release topical ophthalmic delivery system that can also be modified with such polymers disclosed herein.

In accordance with certain embodiments, an ophthalmically acceptable carrier capable of sustained release includes an aqueous suspension at a pH of from about 3 to about 8 and an osmolality of from about 10 to about 400 mOsm/kg containing from about 0.1% to about 6.5% by weight, based on the total weight of the suspension, of a carboxyl-containing polymer prepared by polymerizing one or more carboxyl-containing monoethylenically unsaturated monomers and less than about 5% by weight of a cross-linking agent, such weight percentages of monomers being based on the total weight of monomers polymerized. The carboxy-containing polymer has average particle size of not more than about 50 μm, and in some embodiments, not more than about 30 μm, in equivalent spherical diameter. The polymer is lightly cross-linked to a degree such that although the suspension is administrable in drop form, upon contact of the lower pH suspension with the higher pH tear fluid of the eye, the suspension is gellable to a substantially greater viscosity than the viscosity of the suspension as originally administered in drop form. Accordingly, the resulting more viscous gel can remain in the eye for a prolonged period of time so as to release a medicament contained therein in sustained fashion. These properties remain upon addition of the second polymer to the carboxy-containing aqueous suspension. Without being bound by the theory, the cationic polymer increases the viscosity of the base carboxy-containing aqueous suspension, providing beneficial rheological and mucoadhesive properties.

The carboxy-containing polymer is, in one embodiment, prepared from at least about 50% by weight, and in other embodiments from at least about 90% by weight, of one or more carboxyl-containing monoethylenically unsaturated monomers. The carboxy-containing polymer can be prepared by suspension or emulsion polymerizing acrylic acid and a non-polyalkenyl polyether difunctional cross-linking agent to a particle size of not more than about 50 μm in one embodiment, and not more than about 30 μm, in equivalent spherical diameter, in other embodiments. In one embodiment, the cross-linking agent is divinyl glycol. In other embodiments, up to about 40% by weight of the carboxyl-containing monoethylenically unsaturated monomers can be replaced by one or more non-carboxyl-containing monoethylenically unsaturated monomers containing only physiologically and ophthalmologically innocuous substituents.

The osmolality, in some embodiments, achieved by using a physiologically and ophthalmologically acceptable salt in an amount of from about 0.01% to about 1% by weight, based on the total weight of the suspensions. Exemplary salts include potassium and sodium chlorides and others as defined above.

In some embodiments, in a method of preparing sustained release topical ophthalmically acceptable vehicles, the foregoing suspensions modified with the cationic polymer, are prepared and packaged at the desired viscosity of from 1,000 to about 30,000 cps for administration to the eye in drop form. In one exemplary delivery method, the foregoing suspensions, containing the medicament, are administered to the eye at the initial viscosity in drop form to cause the administered suspension, upon contact with the higher pH tear fluid of the eye, to rapidly gel in situ to a significantly greater viscosity. The more viscous gel remains in the eye for a prolonged period of time so as to release the active ingredient in a sustained fashion.

In contrast to other systems, an ophthalmically acceptable vehicle of the present disclosure not only has the benefit of administration in drop form, but also does not suffer from breakdown limitations due to administration at a viscosity suitable for drops. Through administration at a viscosity such that the suspension can be reliably administered in drop form, but which actually increases when the suspension is so administered, controlled release of the active ingredient is significantly enhanced.

A viscosity substantially over 30,000 cps is not useful for drop formulations; when the viscosity is substantially lower than about 1,000 cps, the ability to gel upon contact with tears can be impeded and ocular retention is reduced. The increased gelation upon contact with the tears occurs with a pH change when a suspension having a pH of from about 3 to about 7.4 and an osmolality of from about 10 to about 400 mOsm/kg, contacts tear fluid, which has a higher pH of about 7.2 to about 8.0. Without being bound by the theory, with the pH increase, the carboxylic acid (COOH) functional group disassociates into carboxylate anions (COO⁻). Through electrostatic interactions, these carboxylate ions repel each other, causing the polymer to expand. The presence of the trace second polymer in the system can provide additional electrostatic, hydrogen bonding, and possible salt-bridge interactions with the mucins of the ocular mucosa, in addition to providing the initial beneficial viscosity modifying properties to the base vehicle. These chemical interactions result in enhanced controlled release of medicament from the vehicle.

The relationship of cross-linking and particle size can be significant. Because the particles are present in a suspension, the degree of cross-linking is necessarily at a level that avoids substantial dissolution of the polymer. On the other hand, since rapid gelation is achieved at the time of the pH change, the degree of cross-linking is necessarily not so great that gelation is precluded. Moreover, if the polymer particle size is too large, induced swelling can tend to take up voids in the volume between large particles that are in contact with one another, rather than the swelling tending to cause gelation.

In a suspension, particle size can be relevant to comfort. However, it has been found that in the system of the present composition, the small particle size and light cross-linking act synergistically to yield the observed rapid gelation when the pH is raised. The use of particles greater than 50 μm eliminates the observed gelation when the pH of the vehicle is increased. Moreover, at the 50 μm size, there is also reasonably good eye comfort.

In some embodiments, the particles are not only subject to the upper size limits described above, but also to a narrow particle size distribution. Use of a monodispersion of particles, which aids in good particle packing, yields a maximum increased viscosity upon contact of the suspension with the tears and increases eye residence time. At least about 80% in some embodiments, at least about 90% in other embodiments, and at least about 95% in still other embodiments, of the particles should be within a no more than about 10 μm band of major particle size distribution, and overall (i.e., considering particles both within and outside such band) there should be no more than about 20%, in some embodiments, and no more than about 10%, in other embodiments, and no more than about 5%, in still other embodiments, fines (i.e., particles of a size below 1 μm. In some embodiments, the average particle size is lowered from an upper limit of 50 μm, such as 30 μm, and to even smaller sizes such as 6 μm, such that the band of major particle size distribution is also narrowed, for example to 5 μm. In some embodiments, sizes for particles within the band of major particle distribution are less than about less than about 20 μm in other embodiments, and from about 1 μm to about 5 μm in still other embodiments.

The lightly cross-linked polymers of acrylic acid or related alpha, beta-unsaturated carboxylic acids used in ophthalmically acceptable vehicle are well known in the art. In one embodiment such polymers are prepared from at least about 90%, or about 95%, or about 99.9% by weight, based on the total weight of monomers present, of one or more carboxyl-containing monoethylenically unsaturated monomers. Acrylic acid is a common carboxyl-containing monoethylenically unsaturated monomer, but other unsaturated, polymerizable carboxyl-containing monomers, such as methacrylic acid, ethacrylic acid, β-methylacrylic acid (crotonic acid), cis-α-methylcrotonic acid (angelic acid), trans-α-methylcrotonic acid (tiglic acid), α-butylcrotonic acid, α-phenylacrylic acid, α-benzylacrylic acid, α-cyclohexylacrylic acid, β-phenylacrylic acid (cinnamic acid), coumaric acid (o-hydroxycinnamic acid), umbellic acid (p-hydroxycoumaric acid), and the like can be used in addition to or instead of acrylic acid.

Such polymers are cross-linked by using a small percentage, i.e., less than about 5%, such as from about 0.5% or from about 0.1% to about 1%, and in other embodiments from about 0.2% to about 1%, based on the total weight of monomers present, of a polyfunctional cross-linking agent. Included among such cross-linking agents are non-polyalkenyl polyether difunctional cross-linking monomers such as divinyl glycol; 2,3-dihydroxyhexa-1,5-diene; 2,5-dimethyl-1,5-hexadiene; divinylbenzene; N,N-diallylacrylamide; N,N-diallylmethacrylamide and the like. Also included are polyalkenyl polyether cross-linking agents containing two or more alkenyl ether groupings per molecule, preferably alkenyl ether groupings containing terminal H₂ C═C<groups, prepared by etherifying a polyhydric alcohol containing at least four carbon atoms and at least three hydroxyl groups with an alkenyl halide such as allyl bromide or the like, e.g., polyallyl sucrose, polyallyl pentaerythritol, or the like; see, e.g., Brown U.S. Pat. No. 2,798,053. Diolefinic non-hydrophilic macromeric cross-linking agents having molecular weights of from about 400 to about 8,000, such as insoluble di- and polyacrylates and methacrylates of diols and polyols, diisocyanate-hydroxyalxyl acrylate or methacrylate reaction products, and reaction products of isocyanate terminated prepolymers derived from polyester diols, polyether diols or polysiloxane diols with hydroxyalkylmethacrylates, and the like, can also be used as the cross-linking agents; see, e.g., Mueller et al. U.S. Pat. Nos. 4,192,827 and 4,136,250.

The lightly cross-linked polymers can be made from a carboxyl-containing monomer or monomers as the sole monoethylenically unsaturated monomer present, together with a cross-linking agent or agents. They can also be polymers in which up to about 40%, and in some embodiments, from about 0% to about 20% by weight, of the carboxyl-containing monoethylenically unsaturated monomer or monomers has been replaced by one or more non-carboxyl-containing monoethylenically unsaturated monomers containing only physiologically and ophthalmologically innocuous substituents, including acrylic and methacrylic acid esters such as methyl methacrylate, ethyl acrylate, butyl acrylate, 2-ethylhexylacrylate, octyl methacrylate, 2-hydroxyethyl-methacrylate, 3-hydroxypropylacrylate, and the like, vinyl acetate, N-vinylpyrrolidone, and the like; see Mueller et al. U.S. Pat. No. 4,548,990 for a more extensive listing of such additional monoethylenically unsaturated monomers. In some embodiments, polymers are lightly cross-linked acrylic acid polymers wherein the cross-linking monomer is 2,3-dihydroxyhexa-1,5-diene or 2,3-dimethylhexa-1,5-diene.

Exemplary commercially available lightly cross-linked carboxy-containing polymers useful in the composition include, for example, polycarbophil (available, for example, from Lubizol, Wichliffe, Ohio), a polyacrylic acid cross-linked with divinyl glycol, Noveon AA-1. Without being bound by theory, this polymer benefits from its mucoadhesive properties which aid in increasing the residence time of the active ingredient in the eye. Other mucoadhesive polymers can be used in conjunction with, or in lieu of the lightly cross-linked polymers disclosed herein, for example, Carbopols such as 934P, 940, 941, 971P, 974P, 980, 981 or hyaluronic acid. The latter has been demonstrated to be an effective mucoadhesive polymer in ocular formulations (Saettone et al. Int. J. Pharm. 51: 203-212, (1989)).

The lightly cross-linked carboxy-containing polymers can be prepared by suspension or emulsion polymerizing the monomers, using conventional free radical polymerization catalysts, to a dry particle size of not more than about 50 μm in equivalent spherical diameter; e.g., to provide dry polymer particles ranging in size from about 1 to about 30 μm, and in other embodiments from about 3 to about 20 μm, in equivalent spherical diameter. In general, such polymers will range in molecular weight estimated to be about 100,000 to about 4,000,000, and in some embodiments, about 2,000,000,000 to about 4,000,000,000.

Aqueous suspensions containing polymer particles prepared by suspension or emulsion polymerization whose average dry particle size is appreciably larger than about 50 μm in equivalent spherical diameter are less comfortable when administered to the eye than suspensions otherwise identical in composition containing polymer particles whose equivalent spherical diameters are, on the average, below about 50 μm. Moreover, above the average 50 μm size, the advantage of substantially increased viscosity after administration is not realized. It has also been discovered that lightly cross-linked polymers of acrylic acid or the like prepared to a dry particle size appreciably larger than about 50 μm in equivalent spherical diameter and then reduced in size, e.g., by mechanically milling or grinding, to a dry particle size of not more than about 50 μm in equivalent spherical diameter do not work as well as polymers made from aqueous suspensions in the ophthalmic vehicle of the present disclosure.

While not being bound by any theory or mechanism advanced to explain the functioning of an ophthalmically acceptable vehicle of the present disclosure, one possible explanation for the difference of such mechanically milled or ground polymer particles as the sole particulate polymer present is that grinding disrupts the spatial geometry or configuration of the larger than 50 μm lightly cross-linked polymer particles, perhaps by removing uncross-linked branches from polymer chains, by producing particles having sharp edges or protrusions, or by producing ordinarily too broad a range of particle sizes to afford satisfactory delivery system performance. A broad distribution of particle sizes impairs the viscosity-gelation relationship. In any event, such mechanically reduced particles are less easily hydratable in aqueous suspension than particles prepared to the appropriate size by suspension or emulsion polymerization, and also are less able to gel in the eye under the influence of tear fluid to a sufficient extent and are less comfortable once gelled than gels produced in the eye using the aqueous suspensions of this vehicle. However, up to about 40% by weight, e.g., from about 0% to over 20% by weight, based on the total weight of lightly cross-linked particles present, of such milled or ground polymer particles can be admixed with solution or emulsion polymerized polymer particles having dry particle diameters of not more than about 50 μm in the ophthalmically acceptable vehicle of the present disclosure. Such mixtures also provide satisfactory viscosity levels in the ophthalmically acceptable vehicle and in the in situ gels formed in the eye coupled with ease and comfort of administration and satisfactory sustained release of the active ingredient to the eye, particularly when such milled or ground polymer particles, in dry form, average from about 0.01 to about 30 μm, and in other embodiments, from about 1 to about 5 μm, in equivalent spherical diameter.

In some embodiments, the particles have a narrow particle size distribution within a 10 μm band of major particle size distribution which contains at least 80%, in other embodiments at least 90%, and in still other embodiments at least 95% of the particles. Also, there is generally no more than about 20%, and in other embodiments no more than about 10%, and in still other embodiments no more than about 5% particles of a size below 1 μm. The presence of large amounts of such fines has been found to inhibit the desired gelation upon eye contact. Apart from that, the use of a monodispersion of particles gives maximum viscosity and an increased eye residence time of the active ingredient in the ophthalmically acceptable vehicle for a given particle size. Monodisperse particles having a particle size of about 30 μm and below are present in some embodiments. Good particle packing is aided by a narrow particle size distribution.

The aqueous suspensions can contain amounts of lightly cross-linked polymer particles ranging from about 0.1% to about 6.5% by weight, and in other embodiments from about 0.5% to about 4.5% by weight, based on the total weight of the aqueous suspension. They can be prepared using pure, sterile water, such as deionized or distilled, having no physiologically or ophthalmologically harmful constituents, and are adjusted to a pH of from about 3.0 to about 6.5, and in other embodiments from about 4.0 to about 6.5, using any physiologically and ophthalmologically acceptable pH adjusting acids, bases or buffers, e.g., acids such as acetic, boric, citric, lactic, phosphoric, hydrochloric, or the like, bases such as sodium hydroxide, sodium phosphate, sodium borate, sodium citrate, sodium acetate, sodium lactate, THAM (trishydroxymethylaminomethane), or the like and salts and buffers such as citrate/dextrose, sodium bicarbonate, ammonium chloride and mixtures of the aforementioned acids and bases.

The second polymer can be any polymer that can enhance the viscosity and mucoadhesive properties of the vehicle where the combination is greater than each individual polymer alone and is also ophthalmically acceptable. Numerous examples of ophthalmically acceptable polymers are disclosed in Wagh et al. Asian J. Pharmaceutics (2008), which is incorporated by reference herein in its entirety. Exemplary second polymers include, without limitation, hydroxyproplymethyl cellulose (HPMC), hydroxypropyl cellulose (HPC), methyl cellulose (MC), hydroxyethyl cellulose (HEC), polyacrylic acid (PAA), polyvinyl alcohol, carbomers, sodium hyaluronate, chitosan, cyclodextrins, polygalacturonic acid, polyitaconic acid, xyloglucan, xanthan gum, gellan gum, polyorthoesters, celluloseacetophthalate, poloxamer 407, polyethyleneimine, and polyethylene oxide. In some embodiments, the second polymer can be a neutral polymer, a cationic polymer, or a second anionic polymer

In particular embodiments, the second polymer can be a cationic polymer. Cationic polymers include any ophthalmically acceptable polyamine polymer capable of modulating the rheological and/or mucoadhesive properties of the vehicle. Such polyamines include, for example, poly-L-lysine (PLL), chitosan, a naturally occurring polysaccharide containing D-glucosamine, polyethyleneimine (PEI), and polyquaternium compounds that include but not limited to Polyquarternium 1, Polyquaternium 7, and Polyquarternium 10. Without being bound by theory, a cationic polymer can impact the vehicle characteristics in at least two different ways. Firstly, the cationic polymer can enhance electrostatic interactions between the carrier and the negatively charged mucins of the corneal epithelium. Such an interaction can confer beneficial mucoadhesive properties to the vehicle. Secondly, the viscosity of the aqueous suspension of the carboxy-containing polymer is increased by the addition of a cationic polymer, even prior to administration to the eye. Again, without being bound by theory, the cationic polyamine polymer can assist in particle aggregation through hydrogen bonding and/or by electrostatic interactions to effectively generate larger molecular weight constructs which increase the aqueous suspension's viscosity. In order to realize the benefits of the added cationic polymer, it should present in an amount that allows the particles of the carboxy-containing polymer to remain suspended, since these advantages are lost upon removal of the carboxy-containing particles from a suspended state. The increased viscosity of the dual cationic polymer/carboxy-containing polymer system can also help counter the effects of the clearance mechanisms in the eye.

In some embodiments, the cationic polymer is chitosan. Chitosan is obtained by deacetylation of chitin and possesses mucoadhesive properties due to electrostatic interaction between positively charged chitosan ammonium groups and negatively charged mucosal surfaces. Chitosan is a linear polysaccharide composed of randomly distributed β-(1-4)-linked D-glucosamine and N-acetyl-D-glucosamine. Chitosan is available with varying degrees of deacetylation (% DA) and is generally produced in a range from between about 60 to about 100% deacetylation. The amino group in chitosan has a pKa value of about 6.5, thus, chitosan is positively charged and soluble in acidic to neutral solution with a charge density dependent on pH and the % DA-value. Chitosan can enhance the transport of polar drugs across epithelial surfaces, and is considered biocompatible and biodegradable.

In some embodiments, chitosan used in the vehicle has a molecular weight in a range from between about 50 kDa to about 100 kDa, including any weights in between, while in other embodiments, chitosan used in the vehicle has a molecular weight in a range from between about 1,000 to about 3,000 kDa, and any weights in between. As shown in the Examples below, the range between about 1,000 kDa and about 3,000 kDa appears to have a larger impact on viscosity of the vehicle, even at very small concentrations of the cationic polymer. In order to achieve comparable viscosities with chitosan alone, solutions of chitosan several orders of magnitude more concentrated have been used, for example, from between about 2% to about 4%.

In the ophthalmically acceptable vehicle of the present disclosure, chitosan or other second polymer is present in an amount ranging from between about 0.01% to about 0.5% when using a cationic polymer having a molecular weight ranging from about 50 kDa to about 100 kDa. The amount of cationic polymer or chitosan can be any amount in between, including about 0.01%, 0.025%, 0.05%. 0.075%, 0.10%, 0.15%, 0.20%, 0.25%, 0.30%, 0.35%, 0.40%, 0.45%, and 0.50% and any amount in between these values. When using higher molecular weight cationic polymers, such as between about 1,000 to about 3,000 kDa, the amount of cationic polymer necessary to achieve favorable viscosities can be substantially reduced. For example, the amount of 1,000 kDa to about 3,000 kDa chitosan can be in a range between about 0.01% and 0.5%, or any amount in between including, for example, 0.01%, 0.015%, 0.020%, 0.025%, 0.030%, 0.035%, 0.040%, 0.045%, 0.05%, 0.1%, 0.15%, 0.20%, 0.25%, 0.30%, 0.35%, 0.40%, 0.45%, and 0.50%.

When formulating the aqueous suspensions, their osmolality will be adjusted to from about 10 mOsm/kg to about 400 mOsm/kg, and in other embodiments, from about 100 to about 300 mOsm/kg, using appropriate amounts of physiologically and ophthalmologically acceptable salts. Sodium chloride can be used as an osmolality adjusting agent to adjust the osmolality of the aqueous suspension to approximate that of physiologic fluid. The amounts of sodium chloride ranging from about 0.01% to about 1% by weight, and in other embodiments from about 0.05% to about 0.45% by weight, based on the total weight of the aqueous suspension, will give osmolalities within the above-stated ranges. Equivalent amounts of one or more salts made up of cations such as potassium, ammonium and the like and anions such as chloride, citrate, ascorbate, borate, phosphate, bicarbonate, sulfate, thiosulfate, bisulfite and the like, e.g., potassium chloride, sodium thiosulfate, sodium bisulfite, ammonium sulfate, and the like can also be used in addition to or instead of sodium chloride to achieve osmolalities within the above-stated ranges.

The amounts of lightly cross-linked carboxy-containing polymer particles, cationic polymer, the pH, and the osmolality chosen from within the above-stated ranges can be correlated with each other and with the degree of cross-linking to give aqueous suspensions having viscosities ranging from about 1,000 to about 30,000 cps, and in other embodiments from about 5,000 to about 20,000 cps, as measured at room temperature (about 25° C.) using a Brookfield Digital LVT Viscometer equipped with a number 25 spindle and a 13R small sample adapter at 12 rpm. The correlations of those parameters are also such that the suspensions will gel on contact with tear fluid to give gels having viscosities estimated to range from about 75,000 to about 500,000 cps, e.g., from about 200,000 to about 300,000 cps, measured as above, depending on pH as observed, for example, from pH-viscosity curves. This effect is noted by observing a more viscous drop on the eye as a set cast. The cast, after setting, can be easily removed. Alternatively, the viscosity can be from about 1000 to about 5000 cps as measured with a Brookfield cone and plate viscometer DV-II+ with the spindle no. CP-52 at 6 rpm.

In some embodiments, the viscosity is in a range from about 1,000 to about 30,000 cps, and in other embodiment from about 5,000 to about 20,000 cps. In yet other embodiments, the viscosity is in a range from about 10,000 to about 15,000 cps. The viscosity range can also be between about 1,000 and 5,000 cps, including 1,000, 1,500, 2,000, 2,500, 3,000, 3,500, 4,000, 4500, and 5,000 cps and all values in between. The viscosity range can also be between about 5,000 to about 10,000 cps, including 5,000, 5,500, 6,000, 6,500, 7,000, 7,500, 8,000, 8,500, 9,000, 9,500, and 10,000 cps and all values in between. The viscosity range can also be between about 10,000 to about 15,000 cps, including 10,000, 10,500, 11,000, 11,500, 12,000, 12,500, 13,000, 13,500, 14,000, 14,500, and 15,000 cps and all values in between. The viscosity range can also be between about 15,000 to about 20,000 cps, including 15,000, 15,500, 16,000, 16,500, 17,000, 17,500, 18,000, 18,500, 19,000, 19,500, and 20,000 cps and all values in between. The viscosity range can also be between about 20,000 to about 30,000 cps, including 20,000, 21,000, 22,000, 23,000, 24,000, 25,000, 26,000, 27,000, 28,000, 29,000, and 30,000 cps and all values in between. In some embodiments, the ophthalmically acceptable vehicle can include a thickening agent or viscosfier that modulates the viscosity of the vehicle. These include, without limitation, polyethylene glycols, polyvinyl alcohol, polyacrylic acid, polyethylene oxide, and poloxamers.

In some embodiments, an effective amount of a medicament is used for the treatment of an ocular condition following surgery. An effective amount will achieve the goal of preventing, reducing, or eliminating the ocular condition. An effective amount includes from about 1 μg to 10,000 μg per dose in one embodiment, and from about 100 μg to 1000 μg per dose in another embodiment. An effective amount includes all values in between and fractions thereof, for example, about 0.1 μg, 100+1 μg and up to about 10000 μg per dose. An effective amount can administered in a dosing regimen once per day, twice per day, thrice per day, or any number of times per day and can be determined in consultation with a physician. An effective amount can be administered as a solution in eye drop form as about a 0.05% to about 5.0% by weight solution of the active ingredient, including for example, about 0.05%, 0.06%, 0.07%, 0.08%, 0.09%, 0.10%, 0.11%, 0.12%, 0.13%, 0.14%, 0.15%, 0.16%, 0.17%, 0.18%, 0.19%, 0.20%, 0.25%, 0.30%, 0.35%, 0.40%, 0.45%, 0.50%, 0.60%, 0.75%, 1.0%, 1.5%, 2.0%, 2.5%, 3.0%, 3.5%, 4.0%, 4.5%, and 5.0%, and all values in between and fractions thereof.

In some embodiments, a medicament, a substance used in treating or ameliorating a disease or medical condition, including drugs intended to treat therapeutically the eye itself or the tissues surrounding the eye and drug administered via the ophthalmic route to treat therapeutically a local condition other than one involving the eye, will typically be incorporated in the ophthalmically acceptable vehicle in therapeutically active amounts comparable to amounts administered in other dosage forms, usually in amounts ranging from about 0.005% to about 10% by weight, and preferably from about 0.01% to about 5% by weight, based on the total weight of the formulation. Thus, for example, from about 0.01% to about 1% by weight of the anti-inflammatory steroid fluorometholone can be administered in this manner.

In some embodiments the corticosteroid or an ophthalmically acceptable salt thereof is present in a range from about 0.05% to about 5.0% by weight, while in other embodiments the active ingredient is present in a range from about 0.05% to about 1%, e.g., from about 0.08% to about 0.0.5% by weight. The amount of active ingredient based on weight percent can be any value between these values, including for example, 0.05%, 0.060%, 0.07%, 0.08%, 0.09%, 0.10%, 0.11%, 0.12%, 0.13%, 0.14%, 0.15%, 0.16%, 0.17%, 0.18%, 0.19%, 0.20%, 0.25%, 0.30%, 0.35%, 0.40%, 0.45%, 0.50%, 0.60%, 0.75%, 1.0%, 1.5%, 2.0%, 2.5%, 3.0%, 3.5%, 4.0%, 4.5%, and 5.0% by weight and all values in between and fractions thereof.

The viscous gels that result upon administration of the aqueous suspensions of the present disclosure to the eye have residence times in the eye ranging from about 2 to about 12 hours, e.g., from about 3 to about 6 hours. The active ingredients contained in these ophthalmically acceptable vehicles are released from the gels at rates that depend on such factors as the active ingredient itself and its physical form, the extent of drug loading and the pH of the system, as well as on any drug delivery adjuvants, such as ion exchange resins compatible with the ocular surface, which can also be present. For fluorometholone, for example, release rates in the rabbit eye in excess of four hours, as measured by fluorometholone contained in the aqueous humor, have been observed.

The active ingredient-ophthalmically acceptable vehicle can be formulated in any of several ways. For example the active ingredient, lightly cross-linked polymer particles, and osmolality-adjusting agent can be pre-blended in dry form, added to all or part of the water, and stirred vigorously until apparent polymer dispersion is complete, as evidenced by the absence of visible polymer aggregates. Sufficient pH adjusting agent is then added incrementally to reach the desired pH, and more water to reach 100 percent formula weight can be added at this time, if necessary. Another convenient method involves adding the drug to about 95 percent of the final water volume and stirring for a sufficient time to saturate the solution. Solution saturation can be determined in any known manner, e.g., using a spectrophotometer. The lightly cross-linked polymer particles and the osmolality-adjusting agent are first blended in dry form and then added to the drug-saturated suspension and stirred until apparent polymer hydration is complete. Following the incremental addition of sufficient pH adjusting agent to reach the desired pH, the remainder of the water is added, with stirring, to bring the suspension to 100 percent formula weight.

These aqueous suspensions can be packaged in preservative-free, single-dose non-reclosable containers. This permits a single dose of the active ingredient to be delivered to the eye one drop at a time, with the container then being discarded after use. Such containers eliminate the potential for preservative-related irritation and sensitization of the corneal epithelium, as has been observed to occur particularly from ophthalmic medicaments containing mercurial preservatives. Multiple-dose containers can also be used, if desired, particularly since the relatively low viscosities of the aqueous suspensions of the present disclosure permit constant, accurate dosages to be administered dropwise to the eye as many times each day as necessary.

In those vehicles where preservatives are to be included, suitable preservatives are chlorobutanol, Polyquat, benzalkonium chloride, cetyl bromide, benzethonium chloride, cetyl pyridinium chloride, benzyl bromide, EDTA, phenylmercury nitrate, phenylmercury acetate, thimerosal, merthiolate, acetate and phenylmercury borate, chlorhexidine, polymyxin B sulphate, methyl and propyl parabens, phenylethyl alcohol, quaternary ammonium chloride, sodium benzoate, sodium proprionate, sorbic acid, and sodium perborate. In particular embodiments, the preservative includes benzalkonium chloride.

In some embodiments, the preservative is present in a range from about 0.001 to about 0.02% by weight. The preservative can be present at about 0.001, 0.002, 0.003, 0.004, 0.005% and any amount in between these amounts. In particular, the present disclosure has the benefit of substantial reduction in the use of a bactericidal component. Thus, in some embodiments, the present disclosure provides an ophthalmically acceptable vehicle having less than about 0.01% of a preservative with bactericidal activity in one embodiment, and less than about 0.01%, 0.009%, 0.008%, 0.007%, 0.006%, 0.005%, 0.004%, 0.003%, or 0.002%, in other embodiments.

In some embodiments, the ophthalmically acceptable vehicle includes a wetting agent. Such agents can be useful in distributing the active ingredient in an otherwise predominantly aqueous environment. Such wetting agents include, for example, Poloxamer 407, a triblock copolymer consisting of a central hydrophobic block of polypropylene glycol flanked by two hydrophilic blocks of polyethylene glycol. Other wetting agents that can be used include carboxymethylcellulose, hydroxypropyl methylcellulose, glycerin, mannitol, polyvinyl alcohol, Octoxynol 40 and hydroxyethylcellulose.

The composition containing a medicament and an ophthalmically acceptable vehicle can be individually packaged for a single dose administration; e.g., in a bottle, jar, ampoule, tube, syringe, envelope, container, unit dose container or vial. When the composition is individually packaged, in some embodiments, the composition does not include a preservative. Alternatively, the composition can be contained in a package that is capable of holding multiple units; e.g., in resealable glass or plastic eyedropper bottles.

### EXAMPLES

The following examples are intended to further illustrate certain preferred embodiments of the invention and are not limiting in nature. Those skilled in the art will recognize, or be able to ascertain, using no more than routine experimentation, numerous equivalents to the specific substances and procedures described herein.

### Example 1

In this example, a phase 3 study was carried out as a randomized, double masked, parallel-group, comparative study to evaluate clinical efficacy and safety of certain Investigational Medicinal Products. During this study it was surprising discovered that a corticosteroid, e.g. dexamethasone, or an ophthalmically acceptable salt thereof could be administered in an effective amount to treat inflammation without the concomitant adverse effects of IOP even for treatment periods of two weeks.

Table 1 below provides a formulation of the glucocorticoid dexamethasone as a 0.1% wt/wt solution in the ophthalmically acceptable vehicle DuraSite®. This formulation was used as one of the Investigational Medicinal Products in the study.

TABLE 1

| INGREDIENT | CONCENTRATION (% W/W) |
|---|---|
| Dexamethasone, USP | 0.10 |
| Mannitol, USP | 1.0 |
| Citric Acid Anhydrous, USP | 0.20 |
| Sodium Citrate Dihydrate, USP | 0.14 |
| Poloxamer 407, NF | 0.20 |
| Benzalkonium Chloride, NF | 0.003 |
| Polycarbophil, USP | 0.90 |
| Sodium Chloride, USP | 0.45 |
| Edetate Disodium Dihydrate, USP | 0.10 |
| Sodium Hydroxide, 2N | Adjust to pH 6.3 |
| Water For Injection, USP | q.s. to 100% |

For this study, subjects were instructed to wash their hands thoroughly and then place one drop of the Investigational Medicinal Product (IMP) on their fingertip and smear onto their entire eyelid twice daily (b.i.d.) at approximately 12-hour intervals for 14 days. If both eyes were inflamed, use of the IMP was on both eyelids. Otherwise, use of the IMP was on the study eyelid.

IOP measurements were performed with an applanation tonometer using combination anesthetic/coloring agent (e.g. FLURESS®) or a topical anesthetic (e.g. proparacaine, benoxinate, etc.) plus a coloring agent (fluorescein). Pressure was recorded as mmHg. Measurements of the IOP were conducted following assessment of visual acuity and slit lamp biomicroscopy. When measured in both eyes, one measurement of IOP was obtained and recorded for the right eye and the procedure was repeated for the left eye.

Table 2 provides the IOP data for subject eyes using the vehicle DuraSite® alone and dexamthasone in DuraSite® (Table 1 formulation). The IOP change in the table below was measured as the difference between the IOP of subject eyes after a two week period of twice daily administration of the IMP and at baseline.

TABLE 2

| Product | No. Eyes | IOP Change |
|---|---|---|
| DuraSite ® alone | 277 | 0.07 |
| Dexamethasone in DuraSite ® | 533 | −0.09 |
| (Table 1 formulation) | | |

P-Value of 0.40

As shown by the data in Table 2, the composition of dexamethasone in DuraSite® did not cause a statistically significant elevation in intraocular pressure after administering the composition twice daily over a two week period. Based on this study, it is expected that subjects can be treated after ocular surgery by administering compositions of the present disclosure at least one, two, three to four times a day or more for a period of at least one, two, four, six weeks or longer without adversely elevating IOP to any significant level.

### Example 2

In a second study, a group of subjects were administered belpharoconjunctivitis dosing b.i.d. into the eye with the DuraSite® dexamethasone formulation shown in Table 1. The IOP data are shown in Table 3 below:

TABLE 3
IOP Change belpharoconjunctivitis study

| Visits (IOP Dif.) | Number of Eyes | IOP Change |
|---|---|---|
| V4 − V1 | 252 | 0.46 |

The IOP Dif. provided in Table 3 was measured as the difference between the IOP of subject eyes after a two week period of twice daily administration of the DuraSite® dexamethasone formulation (V4) and at baseline (V1). This data shows that also on dosing for two weeks, no change within the experimental error of the method was observed. This data also illustrates that no statistically significant change was observed for the DuraSite® dexamethasone formulation when compared to published data of an 3-4 mmHg rise in IOP when dexamethasone was administered without the benefit of a sustained release vehicle. Based on this study and literature studies, it is expected that subjects can be treated following ocular surgery by administering compositions of the present disclosure at least one, two, three to four times a day or more for a period of at least one, two, four, six weeks or longer without adversely elevating IOP to any significant level.

Additional exemplary compositions that are useful for practicing the methods of the present disclosure are provided in Table 4 below.

TABLE 4
(2% Compositions)

| Component | 1 | 2 | 3 | 4 | 5 | 6 | 7 | 8 |
|---|---|---|---|---|---|---|---|---|
| Polycarbophil | 0.9 | 0.95 | 0.95 | 0.9 | 0.9 | 0.95 | 0.9 | 0.95 |
| Dexamethasone | 0.1 | 0.1 | | | | 0.1 | | 0.1 |
| Prednisolone | | | 1.0 | 1.0 | | | | |
| acetate | | | | | | | | |
| Fluoro- | | | | | 0.1 | 0.1 | | |
| methalone | | | | | | | | |
| Difluprednate | | | | | | | 0.05 | 0.05 |
| Hydrochloric | | 1.5 | 1.5 | | | 1.5 | | 1.5 |
| Acid 2N | | | | | | | | |
| Chitosan | | 0.025 | 0.025 | | | 0.025 | | 0.025 |
| Poloxamer 407 | 0.02 | 0.02 | 0.02 | 0.02 | 0.02 | 0.02 | 0.02 | 0.02 |
| Sodium Edetate | 0.1 | 0.1 | 0.1 | 0.1 | 0.1 | 0.1 | 0.1 | 0.1 |
| Sodium Citrate | 0.2 | | | 0.2 | 0.2 | | 0.2 | |
| Citric Acid | 0.14 | | | 0.14 | 0.14 | | 0.14 | |
| Sodium | 0.45 | 0.35 | 0.25 | 0.45 | 0.45 | 0.35 | .45 | 0.35 |
| Chloride | | | | | | | | |
| Mannitol | 1.0 | 1.0 | 1.0 | 1.0 | 1.0 | 1.0 | 1.0 | 1.0 |
| Benzalkonium | 0.003 | 0.003 | 0.003 | 0.003 | 0.003 | 0.003 | 0.003 | 0.003 |
| chloride | | | | | | | | |
| Sodium | qs to | qs to | qs to | qs to | qs to | qs to | qs to | qs to |
| Hydroxide | 6.3 | 6.3 | 6.3 | 6.3 | 6.3 | 6.3 | 4-6 | 4-6 |
| Water, USP | qs to | qs to | qs to | qs to | qs to | qs to | qs to | qs to |
| | 100% | 100% | 100% | 100% | 100% | 100% | 100% | 100% |

Formulations 1-8 in Table 4 can be made by adding polycarbophil, sodium chloride and edetate to water by stirring for 0.5 hours. The solution is then sterilized at 121° C. for 45 minutes and cooled to room temperature. The citrate buffer is dissolved in water and added by sterile addition through a 0.2 μm filter while mixing. The mannitol, poloxamer, and NSAID are dissolved in water and added to the batch by sterile addition. The steroidal anti-inflammatory which has been sterilized by Co-60 radiation is added to the batch by sterile dry particle addition and mixed into the batch. The tromethamine buffer and benzalkonium chloride are dissolved and added by sterile filtration while mixing. Sodium hydroxide is added by sterile addition to adjust the pH to the target value.

For formulations that include chitosan, an aqueous solution of chitosan is prepared using hydrochloric acid and the solution is sterile filtered into the sterilized polycarbophil suspension.

These compositions are examples of compositions of the present disclosure that can be used to treat a subject following surgical trauma and it is expected that the compositions do not cause a statistically significant elevation in intraocular pressure in a statistically significant number of subject eyes when administered twice daily over a period of two weeks.

Only the preferred embodiment of the present invention and examples of its versatility are shown and described in the present disclosure. It is to be understood that the present invention is capable of use in various other combinations and environments and is capable of changes or modifications within the scope of the inventive concept as expressed herein. Thus, for example, those skilled in the art will recognize, or be able to ascertain, using no more than routine experimentation, numerous equivalents to the specific substances, procedures and arrangements described herein. Such equivalents are considered to be within the scope of this invention, and are covered by the following claims.

1. A method of treating a subject after ocular surgery, the method comprising administering to an eye of a subject in need thereof a composition comprising at least one corticosteroid or an ophthalmically acceptable salt thereof in an ophthalmically acceptable vehicle that can provide a sustained release of the at least one corticosteroid or an ophthalmically acceptable salt thereof, wherein the composition does not result in a statistically significant elevation in intraocular pressure in a statistically significant number of subject eyes when administered twice daily over a period of two weeks.

2. The method of claim 1, wherein the composition is administered daily over at least a one week period.

3. The method of claim 1, wherein the composition is administered one to four times a day for a period of one to six weeks.

4. The method of claim 1, wherein the vehicle comprises an aqueous suspension including a carboxy-containing polymer.

5. The method of claim 4, wherein the carboxy-containing polymer is in an amount of about 0.5% to about 1.5% by weight of the composition.

6. The method of claim 4, wherein a viscosity of the composition is in the range of about 1,000 to about 2,000 cps.

7. The method of claim 4, wherein the at least one corticosteroid or an ophthalmically acceptable salt thereof is in an amount of about 0.01% to about 1% by weight of the composition.

8. The method of claim 4, wherein the ocular surgery is among one of the following: cataract surgery, LASIK, or photorefractive surgery.

9. The method of claim 1, wherein the vehicle comprises an aqueous suspension including a carboxy-containing polymer and a cationic polymer.

10. The method of claim 9, wherein the carboxy-containing polymer is in an amount of about 0.5% to about 1.5% by weight of the composition.

11. The method of claim 9, wherein a viscosity of the composition is in the range of about 1,000 to about 2,000 cps.

12. The method of claim 9, wherein the at least one corticosteroid or an ophthalmically acceptable salt thereof is in an amount of about 0.01% to about 1% by weight of the composition.

13. The method of claim 9, wherein the carboxy-containing polymer is a polycarbophil and the cationic polymer is a chitosan.

14. A method of treating a patient after ocular surgery, the method comprising administering to an eye of a subject in need thereof a composition comprising dexamethasone or a pharmaceutically acceptable salt thereof in an ophthalmically acceptable vehicle that can provide a sustained release of the dexamethasone or an ophthalmically acceptable salt thereof, wherein the vehicle comprises an aqueous suspension of a carboxy-containing polymer and the composition does not result in a statistically significant elevation in intraocular pressure in a statistically significant number of subject eyes when administered twice daily over a period of two weeks.

15. The method of claim 14, wherein the vehicle further comprises and a cationic polymer.

16. The method of claim 15, wherein the carboxy-containing polymer is a polycarbophil and the cationic polymer is a chitosan.

17. The method of claim 14, comprising treating the subject for inflammation after cataract surgery as the ocular surgery.

18. The method of claim 17, wherein the carboxy-containing polymer is in an amount of about 0.5% to about 1.5% by weight of the composition.

19. The method of claim 17, wherein a viscosity of the composition is in the range of about 1,000 to about 2,000 cps.

20. The method of claim 17, wherein the dexamethasone or a pharmaceutically acceptable salt thereof is in an amount of about 0.08% to about 0.15% by weight of the composition.

21. The method of claim 17, wherein the composition is administered daily over at least a one week period.

22. The method of claim 17, wherein the composition is administered one to four times a day for a period of one to six weeks.

23. The method of claim 14, wherein the composition is administered daily over at least a one week period.

24. The method of claim 14, wherein the composition is administered one to four times a day for a period of one to six weeks.
